(19) United States
(12) Patent Application Publication
(10) Pub. No.: US 2007/0249900 A1
(43) Pub. Date: Oct. 25, 2007

(54) In vivo device with balloon stabilizer and valve

(75) Inventors: Gordon Wilson, Saratoga, CA (US); Kang-Huai Wang, Saratoga, CA (US)

Correspondence Address: Stevens Law GRP, P O Box 1667, San Jose, CA, 95109, US

(73) Assignee: Capso Vision, Inc., Saratoga, CA (US)

(21) Appl. No.: 11/655,639

(22) Filed: Jan. 19, 2007

Related U.S. Application Data
(60) Provisional application No. 60/760,794, filed on Jan. 19, 2006.

(51) Int. Cl. A61B 1/00

(52) U.S. Cl. 600/116

(57) ABSTRACT

An in vivo imaging system is provided with a capsule having at least one balloon configured to orient the capsule in a consistent orientation relative to an internal organ; at least one valve configured to control the quantity of gas within the at least one balloon; and an imager encased within the capsule.

### BACKGROUND OF THE INVENTION

The invention relates to a camera capsule having a miniature camera for providing images of the digestive tract.

Devices for imaging body cavities or passages in vivo are known in the art and include endoscopes and autonomous encapsulated cameras. Endoscopes are flexible or rigid tubes that pass into the body through an orifice or surgical opening, typically into the esophagus via the mouth or into the colon via the rectum. An image is formed at the distal end using a lens and transmitted to the proximal end, outside the body, either by a lens-relay system or by a coherent fiber-optic bundle. A conceptually similar instrument might record an image electronically at the distal end, for example using a CCD or CMOS array, and transfer other image data as an electrical signal to the proximal end through a cable. Endoscopes allow a physician control over the field of view and are well-accepted diagnostic tools. However, they do have a number of limitations, present risks to the patient, are invasive and uncomfortable for the patient, and their cost restricts their application as routine health-screening tools.

Because of the difficulty traversing a convoluted passage, endoscopes cannot reach the majority of the small intestine and special techniques and precautions, that add cost, are required to reach the entirety of the colon. Endoscopic risks include the possible perforation of the bodily organs traversed and complications arising from anesthesia. Moreover, a trade-off must be made between patient pain during the procedure and the health risks and post-procedural down time associated with anesthesia. Endoscopies are necessarily inpatient services that involve a significant amount of time from clinicians and thus are costly.

An alternative in vivo image sensor that addresses many of these problems is capsule endoscopy. A camera is housed in a swallowable capsule, along with a radio transmitter for transmitting data, primarily comprising images recorded by the digital camera, to a base-station receiver or transceiver and data recorder outside the body. The capsule may also include a radio receiver for receiving instructions or other data from a base-station transmitter. Instead of radio-frequency transmission, lower-frequency electromagnetic signals may be used. Power may be supplied inductively from an external inductor to an internal inductor within the capsule or from a battery within the capsule.

An early example of a camera in a swallowable capsule is described in the State of Israel, Ministry of Defense Pat. No. 5,604,531. A number of patents assigned to Given Imaging describes more details of such a system, using a transmitter to send the camera images to an external receiver. Examples are U.S. Pat. Nos. 6,709,387 and 6,428,469. There are also a number of patents to Olympus describing similar technology. For example, Olympus U.S. Pat. No. 4,278,077 shows a capsule with a camera for the stomach, which includes film in the camera. Olympus U.S. Pat. No. 6,939,292 shows a capsule with a memory and a transmitter.

An advantage of an autonomous encapsulated camera with an internal battery is that measurements may be made with the patient ambulatory, out of the hospital, and with moderate restriction of activity. The base station includes an antenna array surrounding the bodily region of interest and this array can be temporarily affixed to the skin or incorporated into a wearable vest. A data recorder is attached to a belt and includes a battery power supply and a data storage medium for saving recorded images and other data for subsequent uploading onto a diagnostic computer system.

A common diagnostic procedure involves the patient swallowing the capsule, whereupon the camera begins capturing images and continues to do so at intervals as the capsule moves passively through the cavities made up of the inside tissue walls of the GI tract under the action of peristalsis. The capsule's value as a diagnostic tool depends on it capturing images of the entire interior surface of the organ or organs of interest. Unlike endoscopes, which are mechanically manipulated by a physician, the orientation and movement of the capsule camera are not under an operator's control and are solely determined by the physical characteristics of the capsule, such as its size, shape, weight, and surface roughness, and the physical characteristics and actions of the bodily cavity. Both the physical characteristics of the capsule and the design and operation of the imaging system within it must be optimized to minimize the risk that some regions of the target lumen are not imaged as the capsule passes through the cavity.

Two general image-capture scenarios may be envisioned, depending on the size of the organ imaged. In relatively constricted passages, such as the esophagus and the small intestine, a capsule which is oblong and of length less than the diameter passage, will naturally align itself longitudinally within the passage. Typically, the camera is situated under a transparent dome at one (or both) ends of the capsule. The camera faces down the passage so that the center of the image comprises a dark hole. The field of interest is the intestinal wall at the periphery of the image

FIG. 1 illustrates a capsule camera in the prior art. The capsule 100 is encased in a housing 101 so that it can travel in vivo inside an organ 102, such as an esophagus or a small intestine, within an interior cavity 104. The capsule may be in contact with the inner surfaces 106,108 of the organ, and the camera lens opening 110 can capture images within its field of view 112. The capsule may include an output port 114 for outputting image data, a power supply 116 for powering components of the camera, a memory 118 for storing images, image compression 120 circuitry for compressing images to be stored in memory, an image processor 122 for processing image data, and LEDs 126 for illuminating the surfaces 106,108 so that images can be captured from the light that is scattered off of the surfaces.

It is desirable for each image to have proportionally more of its area to be intestinal wall and proportionally less the receding hole in the middle. Thus, a large FOV is desirable. A typical FOV is 140°. Unfortunately, a simple wide-angle lens will exhibit increased distortion and reduced resolution and numerical aperture at large field angles. High-performance wide-angle and “fish-eye” lenses are typically large relative to the aperture and focal length and consist of many lens elements. A capsule camera is constrained to be compact and low-cost, and these types of configurations are not cost effective. Further, these conventional devices waste illumination at the frontal area of these lenses, and thus the power use to provide such illumination is also wasted. Since power consumption is always a concern, such wasted illumination is a problem. Still further, since the intestinal wall within the filed of view extends away from the capsule, it is both foreshortened and also requires considerable depth of field to image clearly in its entirety. Depth of field comes at the expense of exposure sensitivity.

The second scenario occurs when the capsule is in a cavity, such as the colon, whose diameter is larger than any dimension of the capsule. In this scenario the capsule orientation is much less predictable, unless some mechanism stabilizes it. Assuming that the organ is empty of food, feces, and fluids, the primary forces acting on the capsule are gravity, surface tension, friction, and the force of the cavity wall pressing against the capsule. The cavity applies pressure to the capsule, both as a passive reaction to other forces such as gravity pushing the capsule against it and as the periodic active pressure of peristalsis. These forces determine the dynamics of the capsule's movement and its orientation during periods of stasis. The magnitude and direction of each of these forces is influenced by the physical characteristics of the capsule and the cavity. For example, the greater the mass of the capsule, the greater the force of gravity will be, and the smoother the capsule, the less the force of friction. Undulations in the wall of the colon will tend to tip the capsule such that the longitudinal axis of the capsule is not parallel to the longitudinal axis of the colon.

Also, whether in a large or small cavity, it is well known that there are sacculations that are difficult to see from a capsule that only sees in a forward looking orientation. For example, ridges exist on the walls of the small and large intestine and also other organs. These ridges extend somewhat perpendicular to the walls of the organ and are difficult to see behind. A side or reverse angle is required in order to view the tissue surface properly. Conventional devices are not able to see such surfaces, since their FOV is substantially forward looking. It is important for a physician to see all areas of these organs, as polyps or other irregularities need to be thoroughly observed for an accurate diagnosis. Since conventional capsules are unable to see the hidden areas around the ridges, irregularities may be missed, and critical diagnoses of serious medical conditions may be flawed. Thus, there exists a need for more accurate viewing of these often missed areas with a capsule.

FIG. 2 shows a relatively straightforward example where the passage 134, such as a human colon, is relatively horizontal, with the exception of the ridge 136, and the capsule sits on its bottom surface 132 with the optical axis of the camera parallel to the colon longitudinal axis. The ridge illustrates a problematic viewing area as discussed above, where the front surface 138 is visible and observable by the capsule 100 as it approaches the ridge. The backside of the capsule 140, however, is not visible by the capsule lens, as the limited FOV 110 does not pick up that surface. Specifically, the range 110 of the FOV misses part of the surface, and moreover misses the irregularity illustrated as polyp 142.

Three object points within the field of view 110 are labeled A, B, and C. The object distance is quite different for these three points, where the range of the view 112 is broader on one side of the capsule than the other, so that a large depth of field is required to produce adequate focus for all three simultaneously. Also, if the LED (light emitting diode) illuminators provide uniform flux across the angular FOV, then point A will be more brightly illuminated than point B and point B more than point C. Thus, an optimal exposure for point B results in over exposure at point A and under exposure at point C. For each image, only a relatively small percentage of the FOV will have proper focus and exposure, making the system inefficient. Power is expended on every portion of the image by the flash and by the imager, which might be an array of CMOS or CCD pixels. Moreover, without image compression, further system resources will be expended to store or transmit portions of images with low information content. In order to maximize the likelihood that all surfaces within the colon are adequately imaged, a significant redundancy, that is, multiple overlapping images, is required.

One approach to alleviating these problems is to reduce the instantaneous FOV but make the FOV changeable. Patent application 2005/0146644 discloses an in-vivo sensor with a rotating field of view. The illumination source may also rotate with the field of view so that regions outside the instantaneous FOV are not wastefully illuminated. This does not completely obviate the problem of wasteful illumination, and furthermore creates other power demands when rotating. Also, this innovation by itself does not solve the depth of field and exposure control problems discussed above.

Alternatively, the capsule may contain a panoramic imaging system that comprises one or more cameras whose field of view is directed largely perpendicular to all sides of an oblong capsule so that a full 360 deg panoramic field of view is covered. A capsule camera with a panoramic annular lens (PAL) is disclosed in U.S. application Ser. No. ______, filed on Dec. 19, 2007, entitled In Vivo Sensor with Panoramic Camera.

A capsule camera 300 having a panoramic annular lens (PAL) 302, is shown schematically in FIG. 3. The lens 302 has a concentric axis of symmetry and comprises two refractive surfaces and two reflective surfaces such that incoming light passes through the first refractive surface into a transparent medium, is reflected by the first reflective surface, then by the second reflective surface, and then exits the medium through the second refractive surface.

The capsule camera 300 includes LED outputs 304 configured to illuminate outside the capsule onto a subject, such as tissue surface being imaged. The LEDs include LED reflectors 306 configured to reflect any stray LED light away from the lens 302. The purpose of the LED light rays is to reflect off of the tissue surface and into the lens 302 so that an image can be recorded. The reflectors serve to reflect any light from the light source, the LEDs, away from the lens 302 so that only light rays reflected from the tissue surface will be imaged. The LEDs are connected to printed circuit boards PCBs 305 that are connected to each other via a conductor wire or plate 307, distributing power to each LED. The lens 302 is configured to receive and capture light rays 308 that are reflected off of an outside surface, such as a tissue surface, and receives the reflected rays through a first refractor 310. The refracted rays 312 are transmitted to a first reflector 314, which transmits reflected rays 316 onto the surface of a second reflector 318. The second reflector then reflects reflected rays 320 through a second refractor 322, sending refracted rays 324 through opening 326 and into a relay lens system 327.

The system shown is a Cooke triplet relay lens, and it includes a first lens 328 for receiving the refracted rays 324 from the second refractor 322. The first lens focuses the light rays 330 onto a second lens 332. Those focused rays 334 are sent to third lens 336, which focuses rays 338 onto sensor 340. The sensor is mounted on PCB 342, which is connected to the capsule outer walls 344.

The capsule 300 further includes electrical conductor 346 connecting the PCB 342 holding the sensor to the conductor plate or wire 307. The electrical conductor 346 is configured for powering the LEDs 304 through the conductor plate 307 and PCBs 305 that hold the LEDs 304.

The PAL lens 302 produces an image with a cylindrical FOV from a point-of-view on the concentric axis. A relay image system after the PAL lens 302 forms an image on a two-dimensional light sensor 340 that may be a commonly known sensor such as a CMOS or CCD array. FIG. 3a illustrates a Cooke triplet relay lens 327. There exists other configurations that are well known in the art and include double-Gauss configurations.

A capsule camera with a panoramic imaging system comprising multiple cameras with overlapping fields of view is disclosed in co-pending and commonly assigned U.S. application Ser. No. ______ filed on Jan. 19, 2007, entitled System and Method for In Vivo Imager with Stabilizer, and illustrated in FIG. 4. FIG. 4 illustrates 2 cameras 404, 406 that share a common image plane 408, but through the action of prisms 410 that fold the optical axes of each camera, have FOVs 409 that are substantially perpendicular to the longitudinal axis 411 of the camera. By combining a sufficient number of such cameras, such as four, the FOVs 409 may overlap so that a full 360 deg FOV about the capsule is covered. Adventitiously, the cameras may share a common image sensor 408 since the images are coplanar, and each can transfer images on their respective sensor areas 418, 420. The image sensor is configured to receive images projected on it by prisms 410, 412 and 414,416 onto image space 418,420. Image processor 422 is configured to process the images using well known processing techniques, such as storage and other processes. Image compressor 424 is configured to compress images so that less information and thus less power is required to transmit the image data. Memory 426 is for storing image data, power 428 is typically a battery for powering the components, and input/output is configured for sending image data and possibly receiving relevant data.

Because panoramic imaging systems capture images of an organ with a field of view substantially perpendicular to the tissue surface, they more readily obtain high resolution, evenly exposed, images of the organ tissues than do systems whose FOVs are centered in the forward or backward direction. Furthermore, panoramic images are more readily stitched together to form a continuous image because consecutive images captured as the capsule traverses the organ are more similar in terms of both exposure and parallax. Even without utilizing true image stitching, panoramic imaging systems facilitate image processing algorithms that reduce the number of redundant images that are stored in the capsule or transmitted wirelessly from the capsule by comparing consecutive images.

In spite of these advantages, a capsule camera with a panoramic imaging system still encounters a number of challenges in a large organ such as the colon. If the length of the capsule is less than the width of the colon, then the capsule's orientation is not well controlled and it may even tumble as it progresses through the organ. When the capsule's longitudinal axis is not parallel to the longitudinal axis of the colon, the panoramic camera's FOV will not be as nearly perpendicular to the wall of the colon, resulting in increased parallax. Furthermore, even when oriented longitudinally, the capsule will typically not be centered in the lumen so that some portions of it are closer to the camera than others. In order to maintain proper focus over a range of object distances, a number of techniques to increase the depth of field are well known. The F/# of the imaging system may be reduced. However, this reduces the diffraction-limited resolution of the system and also requires more illumination to achieve proper exposure. A mechanism for controlling the focus may be included, but the focus must be controlled independently for different viewing directions. One might utilize a plurality of cameras with different FOVs that each have an autofocus mechanism. However, such an approach will add cost, complexity, and power consumption to the system. Finally, techniques such as “wavefront coding” combine an optical filter with image post-processing to increase the depth-of-field. However, these techniques do add noise to the image during post-processing and thereby reduce the dynamic range.

An additional challenge for a capsule camera in the colon is exposure, which, for a camera without a shutter or settable aperture, becomes a problem of illumination. The side of the capsule that is farthest from the lumen wall must produce substantially more illumination than the side that is closest. While illumination about the capsule is more easily controlled than focus, spurious reflections within the capsule of a bright illumination source are more likely to produce noticeable artifacts in the image. Thus, it is desirable to limit the distance between the capsule and the lumen wall.

Finally, a variable capsule-to-tissue distance means that a frame capture rate sufficient to minimize the chance of missing tissues that are close to or touching the capsule will typically result in images of tissues that are farther from the capsule containing redundant information in consecutive images.

All of the aforementioned problems are mitigated if the capsule is maintained in the center of the colon with an orientation aligned to its direction of motion along the colon. One means of stabilizing the colon is disclosed in US patent application US2006/0178557 which describes a capsule with sacks of clay attached to either end. These sacks are covered with a smooth sacrificial layer when the capsule is swallowed, and the sacrificial layer remains intact until dissolved by the action of bacteria upon entering the colon, at which time the clay absorbs water and expands. The overall shape of the system is thus like a dumbbell and the central cylinder of the capsule is suspended in the center of the colon. The application suggests that a plurality of cameras be included in the capsule, each with a different orientation, so that a 360 deg FOV is covered.

While such a system could effectively stabilize the capsule, it has a number of shortcomings. First, a viable means of panoramic imaging is not disclosed. Given the space constraints, no more than one, or at most two, independent conventional cameras can be fit into the capsule. A system that utilizes the expansion of clay upon hydration also suffers from some potential safety issues. First, if the sacks expand prematurely in the small bowel they may place too much pressure on the organ tissues resulting in eschemia and no means of controlling the size or pressure exerted by the sacks is disclosed. Furthermore, no means of reducing the size of the sacks once they have expanded is disclosed. Thus, they may become stuck behind the ileo-cecal valve, should they deploy accidentally in the small bowel, or behind a constriction in the colon that may exist due to an abnormality, or finally they may be difficult to pass through the rectum out of the body.

Thus there exists a need in the art for a more improved system and method for stabilizing a swallowable capsule camera system for safe and effective in-vivo viewing of internal organs such as the colon that are large relative to the diameter of a capsule that is easily swallowed.

Such systems described in these co-pending and commonly assigned applications however, can be improved with an improved mechanism for controlling the inflation and deflation of the balloons while in operation, taking images from within a patient's GI track. As will be seen below, the invention provides such a system and a method that overcomes the problems of the prior art, and they do so in an elegant manner.

### BRIEF DESCRIPTION OF THE DRAWINGS

FIG. 1 is a drawing of a capsule within a small body organ cavity such as the small intestine according to the invention;

FIG. 2 is a drawing of a capsule within a large body organ cavity such as the large intestine according to the invention;

FIG. 3 is a drawing of a capsule according to the invention;

FIG. 4a is a drawing of a capsule according to the invention;

FIGS. 4c,d are graphs;

FIG. 5 is an embodiment of a plug valve according to the invention;

FIG. 6 is an embodiment of a capsule FIG. 3 is a drawing of a capsule according to the invention;

FIG. 7 is an embodiment of a capsule FIG. 3 is a drawing of a capsule according to the invention;

FIG. 8 is an embodiment of a capsule FIG. 3 is a drawing of a valve according to the invention;

FIGS. 9-10g are flow charts of methods of the invention;

FIG. 11 is a view of two images capture showing overlap; and

FIG. 12 is an example of an image projection from a panoramic camera.

### DETAILED DESCRIPTION

The invention is directed to an in vivo imaging system configured in a capsule for capturing images as the capsule travels through internal organs, such as the gastro intestinal (GI) track. Typically the capsule is cylindrical in shape, with rounded ends much like a vitamin or other pill that can be swallowed. Such a capsule can be swallowed by a patient for examination, and the capsule can capture images with its built-in imager, such as a camera, as it travels through the GI track. In smaller organs, such as the esophagus or the small intestine, the capsule can be relatively easily oriented, where the capsule can maintain a steady orientation while traveling through these smaller organs. In larger organs, however, the orientation can become unsteady, where the focal distance between the imager and the tissue being observed can widely vary, and the images can become distorted when capture and later observed by a doctor or other medical professional. Thus, stability is needed in larger organs.

For stability in larger organs, such as the large intestine, the invention provides a novel feature, where capsule has at least one expandable balloon configured to orient the capsule in a consistent orientation relative to an internal organ. In one embodiment, the capsule has two expandable balloons located at opposite ends of the capsule. Thus, when the capsule enters an organ that has an internal open space that is large relative to the size of the capsule, the balloons can be expanded to better fit the space. Different embodiments of balloons can be configured for this purpose, and several such configurations are possible. For example, co-pending U.S. patent application Ser. No. ______, filed Dec. 19, 2006, discloses several embodiments of capsules having expandable balloons. These balloons, however, require a save and useful means for controlling the inflation and deflation of the balloons. If a capsule were to be deployed and the balloons were expanded in the wrong location, there could be problems with blockage, discomfort, or other injury to the patient, and surgery may even be required. The invention provides a novel method of controlling the inflation and deflation of the balloons. In order for the capsule to operate properly with an expandable balloon, a novel valve system and method are provided to inflate and deflate the balloon while in use.

As described herein, a valve is a broad term that is meant to describe a barrier between two volumes, such as between the inside and outside of the capsule, that can be opened or removed by providing a signal to, or by apply electrical power to or removing electrical power from, an actuator that controls the valve. The valve may help to contain in a vial or other container an expandable material, such as a liquid that can expand into a vapor to inflate the balloons when the valve is opened. Alternatively, a valve may also be used to deflate the balloons at the desired moment or capsule location within the body. Different embodiments of valves are illustrated and described herein, and are intended only as examples, embodiments of the invention, and are not intended to limit the invention. The spirit and scope of the invention is embodied in the appended claims and their equivalents.

In one embodiment, the capsule includes at least one valve configured to control the quantity of gas or other inflating material within the at least one balloon so that it can operate properly, inflating at the correct time, and deflating when desired or needed. For imaging, an imager encased within the capsule for capturing images as the capsule travels through the gastro intestinal (GI) track.

In one embodiment, a deflation valve is configured to deflate the balloon or balloons upon a predetermined event. The event may be a change in pressure, where the deflation valve is configured to deflate the balloons upon the detection of a change in pressure. The invention may include a pressure detector. The deflation valve may be configured to deflate the balloon upon a change in pressure by puncturing a membrane within the valve. The deflation valve may be a normally-open valve, such that the valve is held closed when power is applied to the valve, wherein the balloon is configured to deflate when power is removed and the valve is opened. A particular configuration may include one or more balloons, and may be a pair of balloons located at either end of the capsule. As such, one or more valves may be incorporated into the capsule for deflating purposes. One valve may be used to control the deflation of all balloons if more than one are employed. Alternatively, each balloon may have an independent valve to deflate separately. The particular configuration would depend on the desired features or specifications of a capsule application.

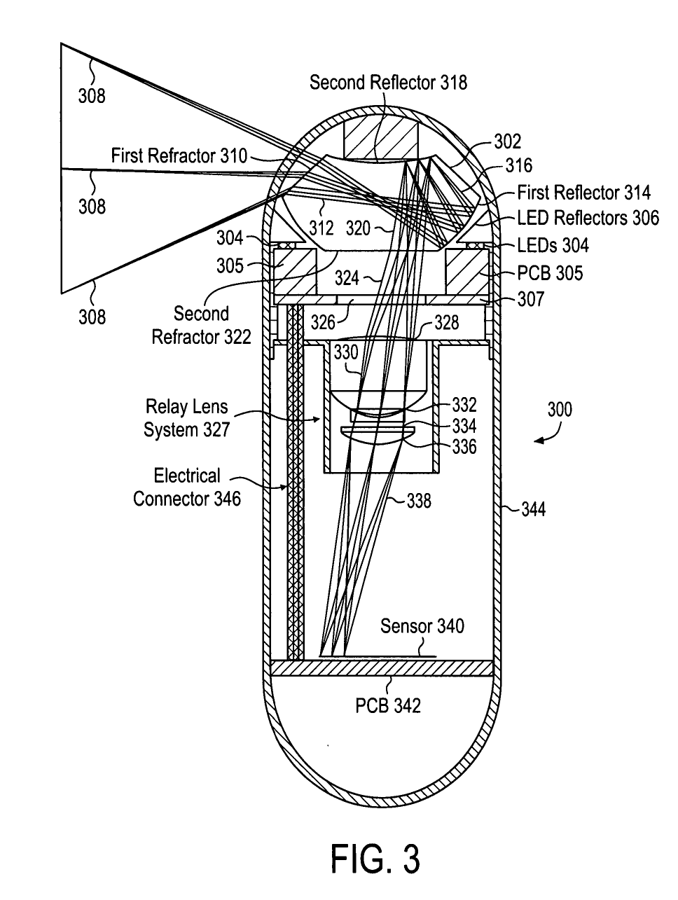

The valve may held closed by a mechanism applied to the valve, wherein the at least one balloon is configured to deflate when the mechanism is removed and the valve is opened. The valve may be held closed by a mechanism applied to the valve, wherein the at least one balloon is configured to deflate when the mechanism is removed and the valve is opened. The deflation valve may include a membrane, such that the membrane seals the balloon closed, wherein the balloon is configured to deflate when the membrane is ruptured. A pair of balloons may be located at opposite ends of the capsule, and at least one release valve may be configured to actuate when a predetermined balloon pressure is detected to deflate the balloons while traveling through an organ. The release valve may be configured to automatically puncture a barrier, such as the membrane of FIG. 3 discussed below, to deflate the balloons upon the occurrence of the predetermined pressure while traveling through an organ.

A capsule may include balloons located at opposite ends of the capsule, a motion detector, and a release valve configured to deflate the balloons when the motion detector determines that the capsule has not progressed significantly for a predetermined period of time. Image processing techniques can be employed in an imaging system design, where movement can be detected by capturing and processing images while moving within an organ. Thus, a motion detector can determine whether the capsule has progressed significantly over the course of some number of sequential image captures.

In another embodiment of the invention, an alternative valve may be used as an inflation valve for expanding a balloon or balloons. The valve may be the same type of valve as the deflation valve, and may even be the same valve in some configurations. It may also be different types of valves with different features or characteristics that are useful and desired in different applications. However, the function of the inflation valve is to connect two or more volumes in order to inflate a balloon or balloon to produce stabilizers for the capsule. The imaging system may have such an inflation valve, and also have one or more balloons configured to expand when the inflation valve is actuated to stabilize the orientation of the capsule while traveling through the internal organ. The inflation valve may be a mechanism configured to release an expansive substance to inflate the at least one balloon when the mechanism is actuated. The expansive substance may be a liquid that expands to a gas, it may be a gas that expands to a larger volume, or it may be a combination of liquid and gas that can expand to inflate the balloons. The mechanism may be a membrane. The capsule may include an electrical element configured to remove the membrane to release a substance to inflate the at least one balloon. The capsule may be configured to capture images while traveling through a gastrointestinal track, where the in vivo camera system operates in a first confined mode while traveling through a smaller organ, such as the small intestine, and in a second expanded mode while subsequently traveling through a large organ, such as through the colon and into the large intestine. The balloon or balloons may be configured to expand when the deflation valve is activated by the occurrence of an event at two ends of the capsule to stabilize the orientation of the capsule while in the large organ.

In one embodiment, the system may include two balloons located at opposite ends of the capsule and configured to inflate at opposite ends of the capsule using a phase transition. This phase transition may be a substance changing from a liquid phase to a gas phase, and may be activated upon the occurrence of an event. The valve may be configured to initiate the phase transition and to inflate the balloons to stabilize the orientation of the capsule. Prior to inflation, the system may include a vial containing a solution such that the total vapor pressure of the solution is substantially equal to a predetermined value. The balloon pressure can increase upon inflation with vapor but will not exceed this predetermined value. The vial may contain a substance that, when released by the valve upon an event, causes the balloon to expand to a predetermined pressure according to the substance characteristics and the balloon architecture. The event may be the detection of passage through the colon using image processing techniques that determine whether images are being captured in a larger cavity, such as in a large organ like the colon. The event may also or alternatively be a predetermined amount of time, the reception of a remote actuation signal, or other event the application calls for.

The system may include at least one reserve configured to store an expandable gas for inflating the balloon. It may also include an electronic balloon actuator configured to cause the valve to release the expandable gas from the reserve to inflate the balloons located at opposite ends of the capsule. The system may include at least one reserve configured to store a mixture of substances that is at least partially in the liquid state, wherein the balloon actuator is configured to cause the valve to release at least one substance from the reserve. This can cause the system to inflate the balloons, perhaps located at opposite ends of the capsule, wherein at least a portion of the substance released vaporizes. The balloons may be configured to inflate at opposite ends of the capsule using a chemical reaction that is activated upon the occurrence of an event to open one or more valves to mix the chemicals in the balloon and initiate the chemical reaction that generates a gas to expand the balloons and to stabilize the orientation of the capsule while moving though an organ.

The balloon attached to a capsule camera should have a means of deflating should the balloon malfunction and inflate in the small bowel prior to passing into the colon. Also, the balloon should deflate should the pressure exerted by the balloon on internal organs exceed a safe limit, either due to a restriction in the organ or a quality-control lapse in balloon manufacturing. The balloon should also deflate whenever the capsule has remained stationary for a certain period of time. This may occur if the balloon is stuck behind the ileo-cecal valve or other constriction or at the rectum. The balloon should also deflate if the capsule loses power, either due to malfunction or battery drain. The deflation can be accomplished by means of a release valve.

According to the invention, a valve for use in an in vivo capsule can have many useful characteristics. In one embodiment the valve is configured to be used only once, such as a membrane that can be punctured or otherwise ruptured at a desired point in the process of traveling through the GI track. In capsule type systems, they are not intended for reuse, particularly given sanitary issues and reliance requirements. The invention provides such a one-use valve mechanism as described herein that is reliable and low cost.

Since most capsule imaging systems are small, there is a need to keep the power low. This way, smaller batteries or other power sources, such as induction power sources, can be used. Thus, the data transmission, data storage (if any) and valve operations must be efficient and have low power consumption. The invention provides different valves that do just that. In one embodiment, an inflation valve is configured as a normally closed valve, where power is required to open the valve to inflate the balloons. In another embodiment, a deflation valve is employed that is normally open, where power is needed to keep the valve closed. This would only need to be closed while the balloons are inflated, and power can be removed when it is desired to deflate the balloons.

A typical capsule endoscope operates with two 1.6V batteries, and, optimally, all capsule functions operate with a supply voltage of 3.2V or less. Otherwise a step-up regulator is required, which is not 100% energy efficient, and adds to the size, cost, and complexity of the system. Thus, a valve with low-voltage actuation is desirable, and the invention provides a means to operate a valve or valves at low voltage as well as at low power.

Since capsule imaging systems are small, there is a need to keep the power consumption low so that small batteries or other power sources, such as induction power sources, can be used. Thus, the valve operations must have low power consumption. Fortunately, the capsule operates in an environment inside the body that exhibits minimal temperature variation, so that a valve may be actuated thermally with minimal power consumption.

In one embodiment, an inflation valve is configured as a normally-closed valve, where power is required to open the valve to inflate the balloons. In another embodiment, a deflation valve is employed that is normally open, where power is needed to keep the valve closed while the balloons are inflated. When power is removed from the valve, it opens and the balloon or balloons deflate.

A typical capsule endoscope operates with two 1.6V batteries, and, optimally, all capsule functions operate with a supply voltage of 3.2V or less. Otherwise a step-up regulator is required, which is not 100% energy efficient, and adds to the size, cost, and complexity of the system. Thus, a valve with low-voltage actuation is desirable, and the invention provides a means to operate a valve or valves at low voltage as well as at low power.

A typical capsule endoscope operates with two 1.6V batteries, and, optimally, all capsule functions operate with a supply voltage of 3.2V or less. Otherwise a step-up regulator is required, which is not 100% energy efficient, and adds to the size, cost, and complexity of the system. Thus, a valve with low-voltage actuation is desirable, and the invention provides a means to operate a valve or valves at low voltage as well as at low power.

Since capsule imaging systems are small, there is a need to keep the power consumption low so that small batteries or other power sources, such as induction power sources, can be used. Thus, the valve operations must have low power consumption. Fortunately, the capsule operates in an environment inside the body that exhibits minimal temperature variation, so that a valve may be actuated thermally with minimal power consumption.

In one embodiment, an inflation valve is configured as a normally-closed valve, where power is required to open the valve to inflate the balloons. In another embodiment, a deflation valve is employed that is normally open, where power is needed to keep the valve closed while the balloons are inflated. When power is removed from the valve, it opens and the balloon or balloons deflate.

A typical capsule endoscope operates with two 1.6V batteries, and, optimally, all capsule functions operate with a supply voltage of 3.2V or less. Otherwise a step-up regulator is required, which is not 100% energy efficient, and adds to the size, cost, and complexity of the system. Thus, a valve with low-voltage actuation is desirable, and the invention provides a means to operate a valve or valves at low voltage as well as at low power.

Since the capsule is limited in size, it is also imperative that the mechanism be very small, so that other components can be located within the capsule. The invention provides such a miniature valve mechanism as described herein.

The particular application of inflating or deflating a balloon attached to an in vivo capsule camera opens up a number of valve design options. Unlike valves in many gas handling applications, this valve need not withstand large pressures or flow rates. Also, linear flow control is not required (the valve requires only an open and a closed state, not intermediary states).

Various MEMS valves exist, for example from Redwood Microsystems. However, these are typically designed for repeat actuation, high pressure, and high flow rates. As such, the power required is too great for the capsule camera application. The fact that the valve only needs to operate once opens up a number of unique design possibilities. One approach would be to create a form of burst valve where an actuator controls the position of a sharp stylus. When the stylus is pushed against a membrane, the membrane bursts, releasing the gas.

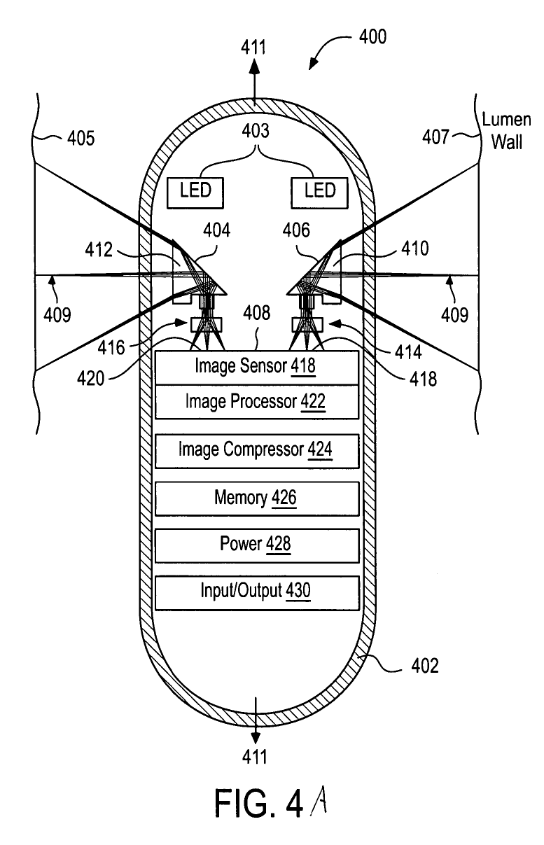

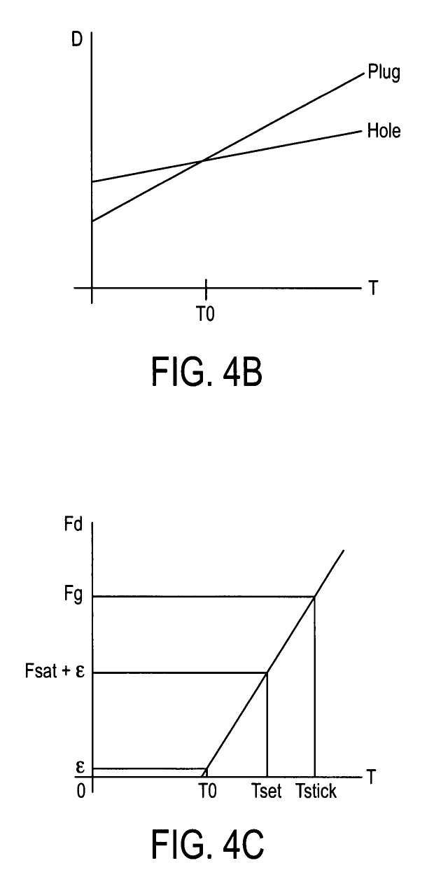

In another embodiment, the release valve consists of a substrate with one or more holes that are filled by plugs. The substrate is made of a material such as glass or ceramic with a low coefficient of thermal expansion (CTE) while the plug is made of a material such as polymer with a higher CTE. The plug and hole may both have a taper. In a preferred embodiment, the taper of the plug exceeds that of the hole. FIG. 4b shows a plot of the diameters of the hole and of the plug in a particular cross sectional plane for each part as a function of temperature, assuming no external force is applied to either the hole or the plug, i.e. when the two parts are not in contact. As the temperature increases, both the material in which the hole is formed and the plug expand. At a particular temperature T0, the diameters are equal. Thus, if the plug is inserted into the hole with both parts at a temperature T0 using an infinitesimal force, the plug will stop at a point where the cross sectional planes defined above for the plug and hole coincide. If a larger force is applied to insert the plug, the plug may be inserted a somewhat greater distance, deforming in the process.

FIG. 4c shows Fd the force required to dislodge the plug once it has been inserted as a function of the plug's temperature T. With increasing temperature, the plug will expand faster than the substrate and the force required to dislodge the plug will increase. The plug may be inserted into the hole during assembly with a pre-set force Fset with the assembly at a particular temperature Tset. In this way, the force needed to dislodge the plug is set—at a temperature Tset, Fd=Fset+ε, where ε is the additional stiction that must be overcome to dislodge the plug. At other temperatures Fd will vary in a monotonic predictable fashion as shown in FIG. 4c. It should be noted that the relationship between Fd and T could vary over time due to stress induced creep in the parts or due to a change in ε the static force of friction (stiction) caused by corrosion, interdiffusion of material, changes in intermolecular forces, static electricity, or chemical reactions. Materials and conditions should be chosen so that these effects are minimized or occur in a predictable fashion.

The temperature of the plug may be raised above ambient by passing a current through a resistive heater proximal to the plug. If the plug has the appropriate electrical resistance, the plug may be heated by directly passing current through it. Polymers exist with a wide variety of electrical conductivities.

The valve is operated in a normally-open mode in that, with no heating current, at operating ambient temperature, and with balloon pressure P1 and ambient pressure P2, where P1>P2, the force Fg=(P1−P2)A exerted on the plug of area A exceeds the holding force and the plug will be ejected. However, with sufficient heating current, the plug heats up and Fg is not sufficient to dislodge the plug. The minimum temperature required to hold the plug under a given pressure differential be referred to as the “stick temperature” Tstick

In order to minimize power consumption while the valve is closed, the CTE difference between the plug and substrate should be as large as possible so that the holding force is a strong function of temperature. Also, the thermal conductivity of the substrate should be low and the plug should be thermally insulated as much as possible. The plug and the heater should have low thermal resistance between them. Tstick must be chosen above the maximum ambient temperature expected during operation. Otherwise the valve will not open. The fact that the range of ambient temperatures for a valve inside the human body is small limits the maximum difference between the stick temperature and the ambient temperature, and thereby the peak power consumption required to keep the valve closed.

In order to ensure that the plug remains in place prior to capsule deployment, when no heating current is applied, a sacrificial holding layer may be applied to the low-pressure side of the substrate. This sacrificial layer should be strong enough to hold the plug in place over the range of shock and vibration that might be experienced during shipment and handling. It may be designed to give way with force Fgas if Fgas exceeds the forces exerted by shock and vibration events. Alternatively, the holding layer may be dissolved by the fluids in the body after the capsule is swallowed. However, allowing liquids to come in contact with the valve will reduce its thermal resistance. The sacrificial layer may be destroyed by other means such as heating, and fluids can be kept out with a vent that comprises a gas permeable membrane that blocks liquid ingress. Alternatively, the plug may adhere to materials on the high pressure side of the plug and these materials may give way under the application of Fgas or may be removed by some means prior to balloon inflation.

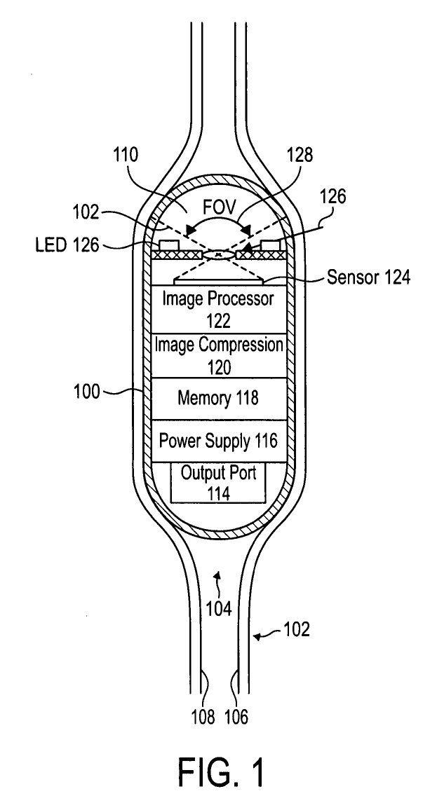

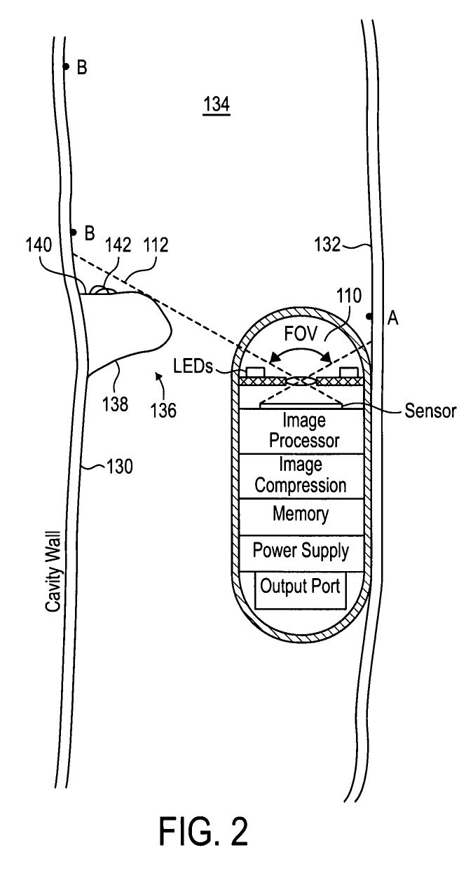

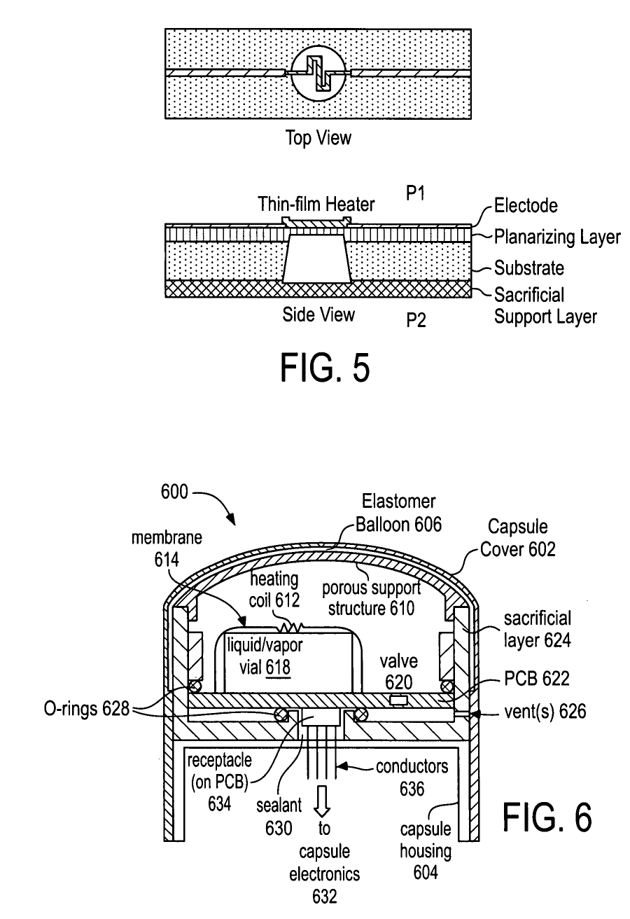

The heater may be a thin film heater on either side of the plug. FIG. 5 shows a heater with electrodes on either side that connect the heater to a source of current. The heater may be made of chromium, tungsten, or some other material of appropriate electrical resistivity. A temperature sensor such as a thermocouple may be included near or on the plug as well. A planarization layer such as spin glass may be placed between the heater and the plug. In FIG. 1 the planarization layer has been etched away around the heater and electrodes so that plug is in direct contact with the gas inside the balloon. Other heater configurations are possible. A thin film heater may be deposited on the inside wall of the hole, for example.

FIG. 6 shows one end of a capsule outfitted with an elastomer balloon. The balloon is stretched over a rounded porous support structure and attached at its edges by means of a pressure fit or epoxy or other means. The balloon and, indeed, the entire capsule may, in turn, be covered with a layer such as gelatin that protects the balloon and minimizes the friction of the capsule during swallowing. The cover material may be chosen to dissolve in stomach acid or to be breached by bacteria in the small or large bowel, prior to balloon inflation. After the cover dissolves bodily fluids may enter the vent and dissolve the sacrificial layer. Current may either be applied to the heater prior to the cover dissolving or else just prior to inflating the balloon.

FIG. 6 shows a balloon inflation mechanism whereby a liquid or mixture of liquids is held in a vial inside the balloon. When current is passed through a resistive heater on the vial, the vial melts at the location of the heater and the liquid vaporizes and inflates the balloon with vapor. The balloon inflation may be actuated by other means such as a chemical reaction that produces a net increase in gas molecules. Alternatively, the balloon may be initially pressurized but constrained by the cover. When the cover dissolves, the balloon may deploy passively, without actuation.

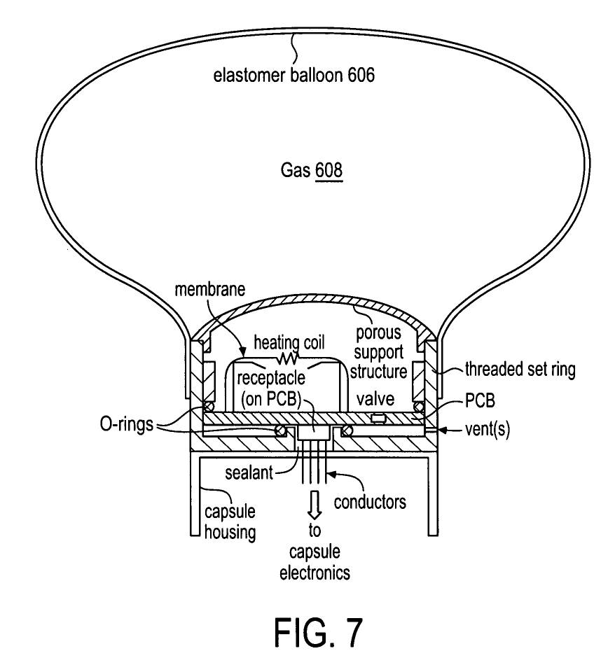

The capsule includes a cover 602 that is to be removed in the process to allow the elastomer balloon to expand. This may be done by breaking away when the balloon is released, or by other means. The capsule includes capsule housing 604, elastomer balloon 606 shown encapsulated in the capsule cover 602. The vapors 608, FIG. 7, expand to inflate the balloon 606 as the cover releases. The porous support structure 610 is configured to support the elastomer balloon in place before deployment, expansion, and also protects the components from the elastomer balloon, so that there is no interference with the components. The housing houses the capsule elements including the sealant for sealing the housing from vapors and other elements, capsule electronics 632, receptacle 634, and conductors 636.

The valve substrate may serve as a printed circuit board (PCB) on which the electrical connections to the balloon actuator reside along with the electrical connections to the valve.

Referring to FIG. 7, an view of the configuration of FIG. 6 is illustrated, where the elastomer balloon is expanded. The capsule cover 602 does not exist on the expanded FIG. 7, because it is removed to allow the balloon to expand.

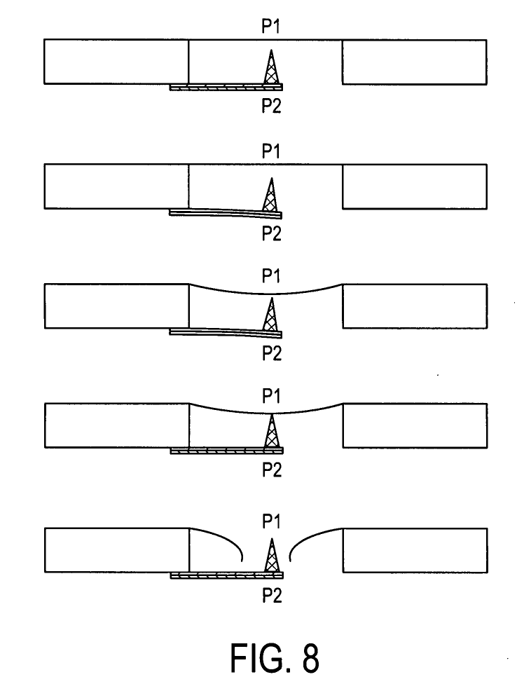

FIG. 8 illustrates another valve. A thin membrane covers a hole separating the balloon at pressure P1 from the environment at pressure P2. Prior to inflation, P1=P2 and the membrane is not deformed. A sharp stylus is attached to a cantilever bimorph actuator, such as a thermal bimorph or piezoelectric bimorph. Other types of actuators could also be used such as electromagnetic, electrostatic, or thermal-mechanical. When actuated, the actuator pulls the stylus away from the membrane. When the balloon inflates, P1>P2 and the membrane deforms toward the stylus. However, if a critical pressure is not exceeded, the membrane will not reach the stylus. Now, if the power to the actuator is removed, the stylus will move into the membrane and burst it so that gas is released from the balloon through the hole.

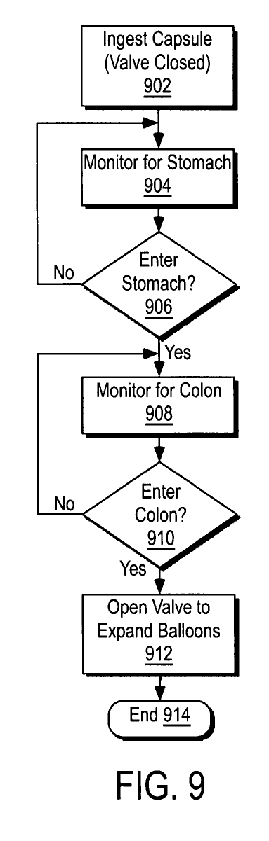

Referring to FIG. 9, one example of a flow process of the capsule is illustrated in terms of events related to the location of the capsule in GI track. In the first step 902, the capsule is ingested. In step 904, it is determined whether the capsule has entered the stomach. If it has not in step 906, the process returns to step 904. If it does, then the event is recorded and the process monitors whether the capsule has entered the colon. If not as determined in step 910, then the process returns to step 908. Once it is in the colon, the process goes to step 912 to open the valve, expanding the balloons. The process ends in step 914.

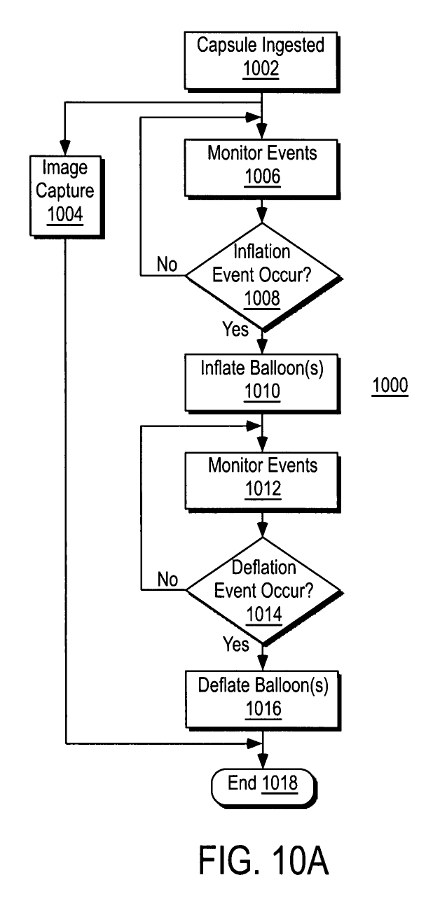

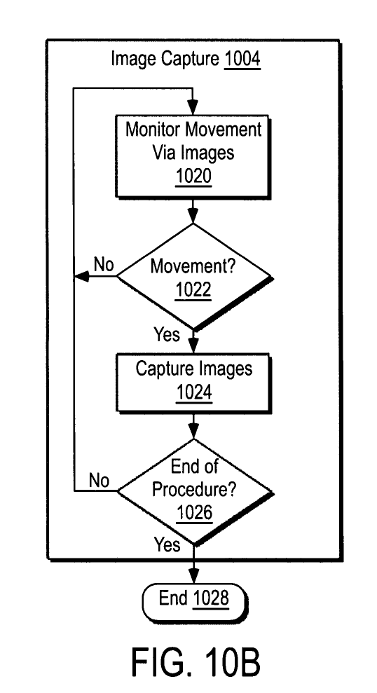

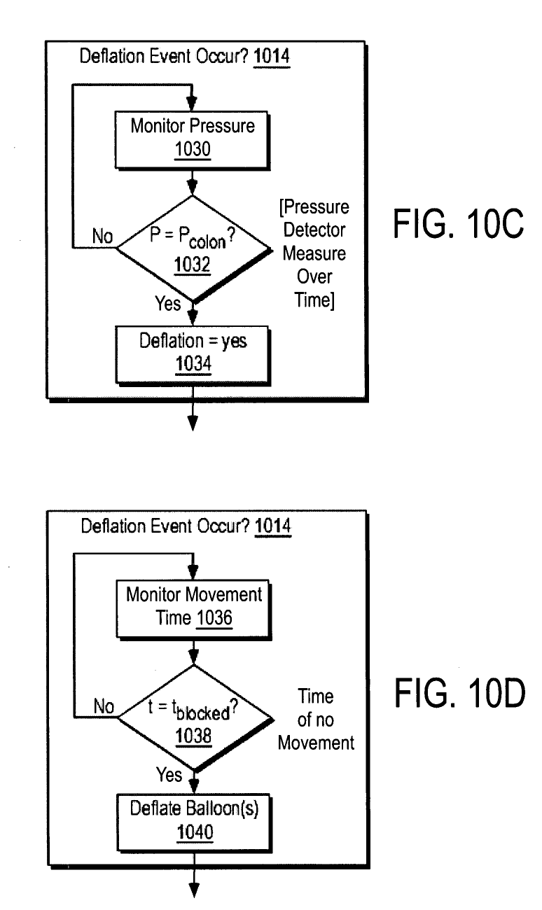

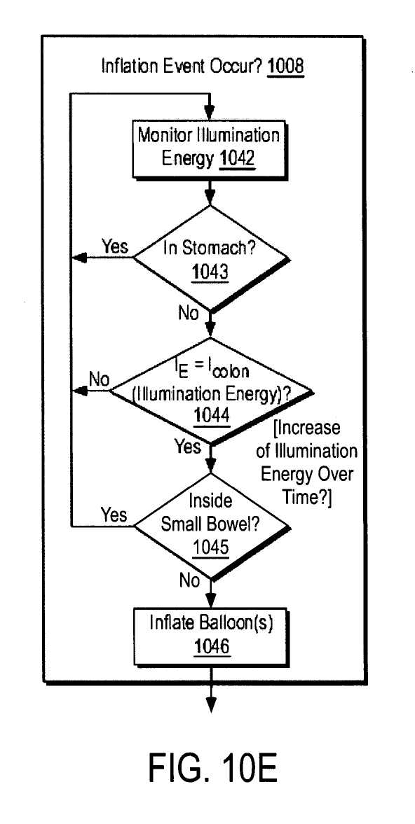

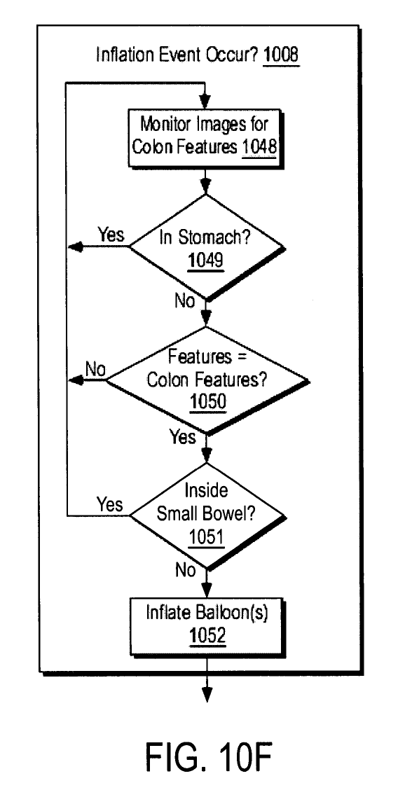

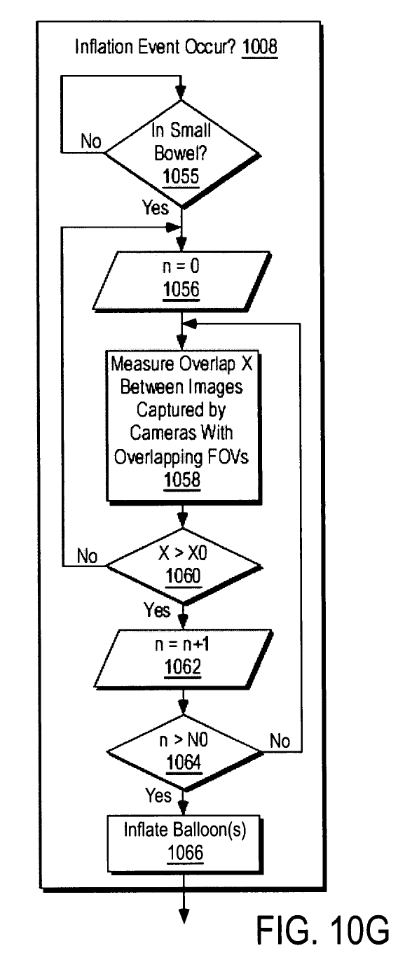

By way of example, one method for inflating and deflating the balloons according to the invention is illustrated in FIG. 10a, a general process 1000 illustrated in flow chart. In operation, a capsule is ingested in step 1002. From there, two processes operate in parallel. In step 1004, image capture occurs, which can occur throughout the process while the capsule travels throughout the GI track. At the same time, a series of monitoring processes occurs beginning with step 1006, where inflation events are monitored. If an inflation event does not occur as determined in step 1008, then the process loops back and continues monitoring the events in step 1006. When an event occurs, then the process initiates the inflation process in 1010, where the balloon or balloons are inflated. After the balloons are inflated, then the process must monitor the system to watch for deflation events in step 1012. Until a deflation event occurs, the process loops back to step 1012, where deflation events continue to be monitored. Once a deflation event occurs as determined in step 1014, then the balloons are deflated in step 1016. The process ends in step 1018.

Referring back, more detailed processes within some of the individual steps of FIG. 10a are illustrated in FIGS. 10b through 10f. In FIG. 10b, a more detailed process of image capture of step 1004 is illustrated. First, the process monitors movement via images in step 1020. Then, it is determined whether there was movement in step 1022. If movement does not occur, then the process loops back to step 1020 for further monitoring. Once movement occurs, then the process proceeds to step 1024, where images are captured. This feature provides for great reduction in images captured, where images are only captured when there is movement, greatly reducing redundant images. Thus, the physician or other medical professional does not need to review as many images as otherwise required. In step 1026, it is determined whether the end of the procedure has been reached. If not, then the process returns to step 1020, where the movement of the capsule is further monitored, and the process continues. If the end of the procedure occurs, whether the capsule has completed the process and been expelled or if it is ended for any other reason, the process ends at step 1028, which corresponds to step 1018 of FIG. 10a.

Referring to FIG. 10c, a more detailed illustration of the step 1014, determining whether a deflation event has occurred, is shown. In step 1030, the pressure is monitored. This process monitors pressure as a deflation event, so that the balloon or balloons would deflate when there is an unsafe increase in pressure, indicating a blockage of some sort, or perhaps a premature inflation in a small organ such as the esophagus or a small intestine, or perhaps the capsule has entered the colon, just before it enters the large intestine, and it is stuck. If no change occurs, the process continues to monitor the pressure in step 1030. If a predetermined pressure level is detected in step 1032, such as P=P_{colon}, this indicates that the capsule has incurred a deflation event in step 1034, and the balloons will be deflated in step 1016 (FIG. 10a).

In FIG. 10d, another embodiment of a determination of whether a deflation event of step 1014 (FIG. 10a) occurs. Here, the time of movement is monitored in step 1036. Here, it is determined in step 1038 whether there has been no substantial movement of the capsule in a person's GI track. If movement occurs, then the process returns to step 1036 for further monitoring. If, however, it is determined in step 1038 that enough time has passed to be concerned, then the process deflates the balloons in step 1040, which corresponds to step 1016 of FIG. 10a. The process then ends in step 1018, FIG. 10a.

Referring to FIG. 10e, an example of a determination of whether an inflation event, step 1008 of FIG. 10a, occurs is illustrated. In step 1042, the illumination energy I_{E} required to obtain a desired image exposure is measured and monitored. In step 1043, it is determined whether the capsule is not in the stomach. If it is in the stomach, the process returns to step 1042 for monitoring. This is useful in preventing premature expansion in the stomach, preventing a false event indication. In step 1044, it is determined whether the illumination energy is at a level that indicates entry of the capsule into the colon, I_{Colon}. If not, the monitoring continues in step 1042. Once such an energy is reached, it is then determined whether the capsule is inside the small bowel in step 1045, this prevents premature inflation as well. If not in the small bowel, then the process returns to step 1042. If it is in the small bowel, then it is not likely a false read. The process then proceeds to the next step where the balloons are inflated in step 1046, corresponding to step 1010, FIG. 10a, and the process proceeds to step 1012.

Referring to FIG. 10f, another example of a determination of whether an inflation event occurs is illustrated. In step 1048, the process monitors images captured for colon features. Then, it is determined whether the capsule is in the stomach. If it is in the stomach, it returns to step 1048. If not in the stomach, the images are then compared in step 1050 to known colon images. If there are no colon images, then the process loops to step 1048 for further monitoring. then determine If an image of a colon does occur in step 1050, then it is determined whether the capsule is in the small bowel. If not in the small bowel, then the process returns to step 1048 for monitoring. If it is in the small bowel, then the process inflates the balloons in step 1052.

Referring to FIG. 10g, the process determines in a different embodiment whether an inflation event occurs. In step 1055, it is determined whether the capsule is in the small bowel. If it is not, then the process goes back until it is in the bowel. Then, the counter is set to zero in step 1056, and the overlap X between images capture by the cameras with overlapping FOVs are measured. In step 1060, it is determined whether the overlap is greater than a predetermine amount X0. If not, the process returns to step 1056. If it does, the counter is incremented in step 1062, and it is determined whether the count exceeds a predetermined count N0. If it does not, the process returns to step 1058. It does exceed N0, then the balloons are inflated in step 1066.

By way of example, FIG. 11 shows a cross section of a cylindrical capsule. Within the capsule are four cameras. These cameras may have separate centers of perspective C1, C2, C3, and C4 that lie in the entrance pupils of each camera. Associated with each camera is also a horizontal field of view HFOV. Each camera “faces” a different direction such that the optical axes are, in this case, separated by 90 deg. Since the HFOV of each camera exceeds 90 deg, the HFOVs overlap. Advantageously, they overlap along vertical lines within the capsule so that the horizontal extent of an object touching the capsule on the outside may be viewed in its entirety.

Also shown in FIG. 11 is a cross section of the lumen. The distances from the center of the capsule O to four points on the lumen wall I, J, K, and L in the plane of cross section are uniquely determined by the amount of overlap between adjacent images captured of the lumen. The distance OK is linearly related to the overlap x.

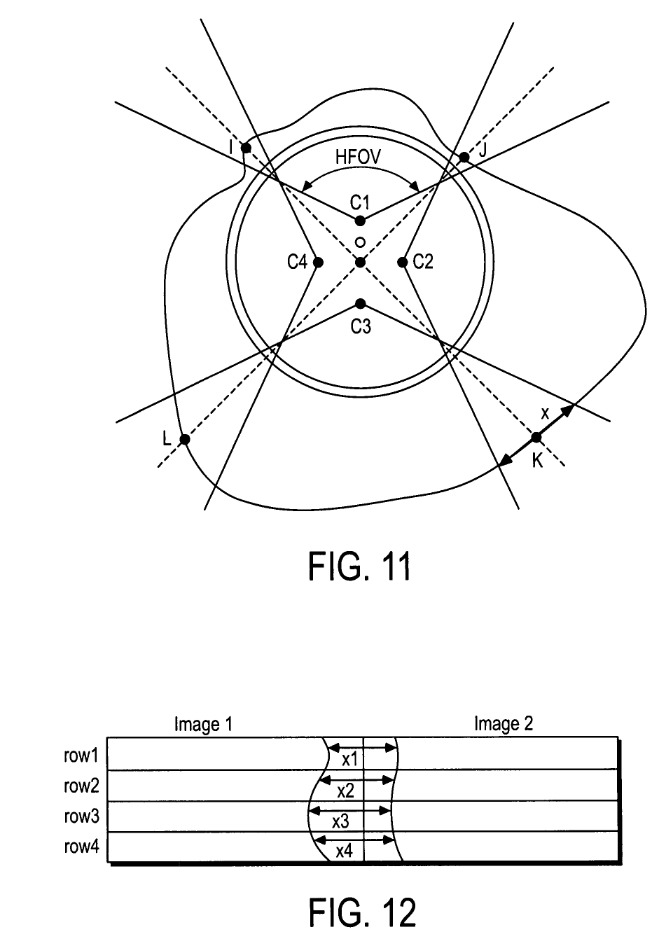

FIG. 12 shows two images captured by two adjacent cameras. The images are placed side-by-side. Only one feature of the images is shown, a line. This line might correspond to the edge of some physical feature on the lumen. Due to the non-coincident centers of perspective and the fact that the line on the lumen is not a constant distance from the capsule along its vertical extent, the line has a slightly different shape in the two images. An algorithm that determines the overlap might first divide the images into a series of horizontal bands (Four are illustrated in FIG. 12). Each band could then be translated horizontally until the best image match is found in the region where the translated bands overlap. In this simple case, that would occur when the line sections most overlap. The optimal translation distances (overlaps) for each band are labeled x1, x2, x3, and x4. Similarly, overlaps and corresponding object distances can be determined at the other three overlap regions. By considering a set of data, an estimate of the cross-sectional area of the lumen can be made. This estimate, along with previous estimates, can then be used to decide whether the capsule has entered the colon and whether to deploy the balloons.

1. An in vivo imaging system comprising:
a capsule having at least one balloon configured to orient the capsule in a consistent orientation relative to an internal organ;
at least one valve configured to control the quantity of gas within the at least one balloon; and
an imager encased within the capsule.

2. An in vivo system according to 1, further comprising a deflation valve configured to deflate the at least one balloon upon a predetermined event.

3. An in vivo system according to claim 2, wherein the event is a change in pressure.

4. An in vivo system according to claim 2, further comprising a pressure detector, wherein the at least one deflation valve is configured to deflate the balloons upon the detection of a change in pressure.

5. An in vivo system according to claim 3, further comprising a pressure detector, wherein the at least one deflation valve configured to deflate the balloons upon a change in pressure by puncturing a membrane of the valve.

6. An in vivo imaging system according to claim 2, wherein the deflation valve is a normally opened valve, such that the valve is held closed when power is applied to the valve, wherein the at least one balloon is configured to deflate when power is removed and the valve is opened.

7. An in vivo imaging system according to claim 2, wherein the deflation valve is a normally opened valve, such that the valve is held closed by a mechanism applied to the valve, wherein the at least one balloon is configured to deflate when the mechanism is removed and the valve is opened.

8. An in vivo imaging system according to claim 2, wherein the deflation valve is a normally opened valve, such that the valve is held closed by a mechanism when power is applied to the valve, wherein the at least one balloon is configured to deflate when power is removed and the valve is opened.

9. An in vivo imaging system according to claim 2, wherein the deflation valve includes a membrane, such that the membrane seals the at least one balloon closed, wherein the at least one balloon is configured to deflate when the membrane is ruptured.

10. An in vivo imaging system according to claim 2, further comprising a pair of balloons located at opposite ends of the capsule, and at least one release valve configured to actuate when a predetermined balloon pressure is detected to deflate the balloons upon the occurrence of the predetermined pressure.

11. An in vivo imaging system according to claim 10, further comprising a release valve configured to puncture a barrier when a predetermined balloon pressure is detected to deflate the balloons upon the occurrence of the predetermined pressure while traveling through an organ.

12. An in vivo imaging system according to claim 10, further comprising a release valve configured to automatically puncture a barrier [membrane of FIG. 3] to deflate the balloons upon the occurrence of the predetermined pressure while traveling through an organ.

13. An in vivo imaging system according to claim 2, further comprising balloons located at opposite ends of the capsule, a motion detector, and a release valve configured to deflate the balloons when the motion detector determines that the capsule has not progressed significantly for a predetermined period of time.

14. An in vivo imaging system according to claim 2, further comprising a motion detector and a release valve configured to deflate the at least one balloon when the motion detector determines that the capsule has not progressed significantly over the course of some number of sequential image captures.

15. An in vivo imaging system according to claim 2, further comprising a release valve configured to deflate the at least one balloon when the motion detector determines that the capsule has not progressed over the course of a predetermined number of sequential image captures.

16. An in vivo imaging system according to claim 1, further comprising an inflation valve, wherein the at least one balloon is configured to expand when the inflation valve is actuated to stabilize the orientation of the capsule while traveling through the internal organ.

17. An in vivo system according to claim 16, wherein the inflation valve is a mechanism configure to release an expansive substance to inflate the at least one balloon when the mechanism is actuated.

18. An in vivo system according to claim 17, wherein the expansive substance is a liquid.

19. An in vivo system according to claim 17, wherein the expansive substance is a gas.

20. An in vivo system according to claim 17, wherein the expansive substance is a combination of liquid and gas.

21. An in vivo system according to claim 17, wherein the mechanism is a membrane.

22. An in vivo system according to claim 21, further comprising an electrical element configured to remove the membrane to release a substance to inflate the at least one balloon.

23. An in vivo imaging system according to claim 16, wherein the capsule is configured to capture images while traveling through a gastrointestinal track, where the in vivo camera system operates in a first confined mode while traveling through the small intestine and in a second expanded mode while subsequently traveling through the colon, wherein the at least one balloon is configured to expand when the deflation valve is activated by the occurrence of an event at two ends of the capsule to stabilize the orientation of the capsule while in the large intestine.

24. An in vivo imaging system according to claim 1, further comprising two balloons located at opposite ends of the capsule and configured to inflate at opposite ends of the capsule using a phase transition that is activated upon the occurrence of an event, where the valve is configured to initiate the phase transition and to inflate the balloons to stabilize the orientation of the capsule.

25. An in vivo imaging system according to claim 24, wherein prior to inflation the system includes a vial containing a solution such that the total vapor pressure of the solution is substantially equal to a predetermined value, such that the balloon pressure upon inflation with vapor will not exceed this predetermined value.

26. An in vivo imaging system according to claim 24, wherein the system further includes a vial containing a substance that, when released by the valve upon an event, causes the balloon to expand to a predetermined pressure according to the substance characteristics and the balloon architecture.

27. An in vivo imaging system according to claim 26, wherein the event is the expiration of a predetermined amount of time.

28. An in vivo imaging system according to claim 26, wherein the event is the reception of a remote actuation signal.

29. An in vivo imaging system according to claim 24, further comprising at least one reserve configured to store an expandable gas and an electronic balloon actuator configured to cause the valve to release the expandable gas from the reserve to inflate the balloons located at opposite ends of the capsule.

30. An in vivo imaging system according to claim 24, further comprising at least one reserve configured to store a mixture of substances that is at least partially in the liquid state, wherein the balloon actuator is configured to cause the valve to release at least one substance from the reserve to inflate the balloons located at opposite ends of the capsule, wherein at least a portion of the substance released vaporizes.

31. An in vivo imaging system according to claim 24, wherein the balloons are configured to inflate at opposite ends of the capsule using a chemical reaction that is activated upon the occurrence of an event to open one or more valves to mix the chemicals in the balloon and initiate the chemical reaction that generates a gas to expand the balloons and to stabilize the capsule.
